(19) United States
(12) Patent Application Publication
(10) Pub. No.: US 2010/0036353 A1
(43) Pub. Date: Feb. 11, 2010

(54) Refusable baby diaper system

(76) Inventor: Sheila Victoria Payne, Buxton, ME (US)

Correspondence Address: Sheila Payne, 81 Allen Ave, Buxton, ME, 04093, US

(21) Appl. No.: 12/462,815

(22) Filed: Aug. 10, 2009

Related U.S. Application Data
(60) Provisional application No. 61/188,506, filed on Aug. 11, 2008.

(51) Int. Cl. A61F 13/511

(52) U.S. Cl. 604/385.08

(57) ABSTRACT

An adjustable, form fitted, reusable diaper which includes a moisture impervious outer cover in a substantially hourglass shape when open, fastens to the wearer around the waist. A reusable detachable moisture pervious topsheet in a substantially hourglass shape attaches to the moisture imperious outer cover. An absorbent layer is placed between the outer cover and the topsheet. The moisture pervious topsheet wicks moisture away from the wearer and into the absorbent layer located between the outer cover and the topsheet. When soiled, the absorbent layer and the topsheet are removed and replaced with additional absorbent layers and topsheets.

### CROSS-REFERENCE TO RELATED APPLICATIONS

This application claims the benefit of provisional patent application Ser. No. 61/188,506, filed Aug. 11, 2008 by the present inventor.

### BACKGROUND

1. Field

This application relates generally to baby diapers and more particularly to reusable cloth diaper systems having multiple fabric layers with removable and washable cloth insert.

2. Prior Art

Parents now have many options in diapering their children, including conventional cloth diaper services, home laundered cloth diapers, single-use paper and polymer diapers, and hybrid diapers that combine a flushable insert with a reusable outer garment. The comparative health, economic, and environmental impacts of these diapering choices has been a source of debate for many years. Ultimately, the specific diapering system chosen depends greatly on the particular needs and concerns of the individual parent, which may change during a child's diapering years or from child to child within a family.

Regardless of the type of diaper chosen, medical experts agree that for a child's health and comfort, a wet or soiled diaper should be changed as soon as possible to minimize skin exposure to the bacteria and chemical compounds present in human waste, thereby preventing diaper rash, urinary tract infections, and other conditions.

Many single-use diapers attempt to address the issue of comfort by utilizing a moisture-pervious topsheet which provides a “feel-dry” barrier to keep the absorbent material out of direct skin contact. Some cloth diapers use similar principles, employing a moisture-pervious fabric such as polyester microfleece, polyester suedecloth, and athletic wicking materials.

Cloth diaper experts have long recommended laying a rectangle of such moisture-pervious fabric as a detached topsheet in a cotton diaper to provide “feel-dry” skin comfort. Using a two-part system has advantages in the thoroughness of cleaning for both the diaper and topsheet, and this allows the moisture impervious outer diaper cover, which is not in direct skin contact, to be reused several times with a new cotton diaper and topsheet installed. However the rectangle shape of the topsheet does not conform to the area where the diaper is worn and such a topsheet will allow the fully saturated cotton diaper to come in contact with the skin. With older, more mobile children, the lay-in topsheet method has significant disadvantages, namely that the movement of the anatomical region in question causes individual pieces of fabric to shift and bunch. Attempting to address that issue, as well as ease of use, prior art example U.S. Pat. No. 6,579,273 attaches the moisture-pervious material to the moisture impervious outer cover by various sewing methods. While achieving the aim of layer stability and ease of use, this system introduces other problems. By permanently attaching the moisture-pervious topsheet to the moisture impervious outer cover, the entire diaper must be changed and washed after each use, causing greater expense due to the larger number of diapers needed as well as decreasing the usable lifespan of each individual diaper. Prior art example U.S. Pat. No. 5,217,447 shows a disposable rectangular moisture-pervious topsheet which is attached to the reusable absorbent pad and the moisture impervious outer cover by means of snap attachments. While this addresses the issue of outer cover lifespan and the number of outer covers needed, it requires the purchase and use of many disposable topsheets, where the overall rectangular shape of which does not conform to the corresponding area where the diaper is to be worn.

### SUMMARY

Accordingly, for those parents who choose cloth diapers as their sole or supplementary diapering system, there is a need for a diapering system which addresses the concerns of customizable absorbency which is easy to launder thoroughly, a moisture-pervious “feel-dry” topsheet which is detachable for ease of cleaning and reusability, as well as conforming to the anatomy of the area covered by a diaper, and an outer moisture-impervious cover that can be reused with a fresh absorbent layer and moisture-pervious topsheet installed.

### DRAWINGS—FIGURES

FIG. 1 is a plan view of the inner surface of the diaper cover

FIG. 2 is a plan view of the outer surface of the diaper cover

FIG. 3 is a plan view of the removable topsheet

FIG. 4 is a plan view of the topsheet assembled into the diaper cover

FIG. 5 is a underside view of the front flap

FIG. 6 is a plan view of the diaper fastened as it would appear on the wearer

FIG. 7 is a side view of the diaper fastened as it would appear on the wearer

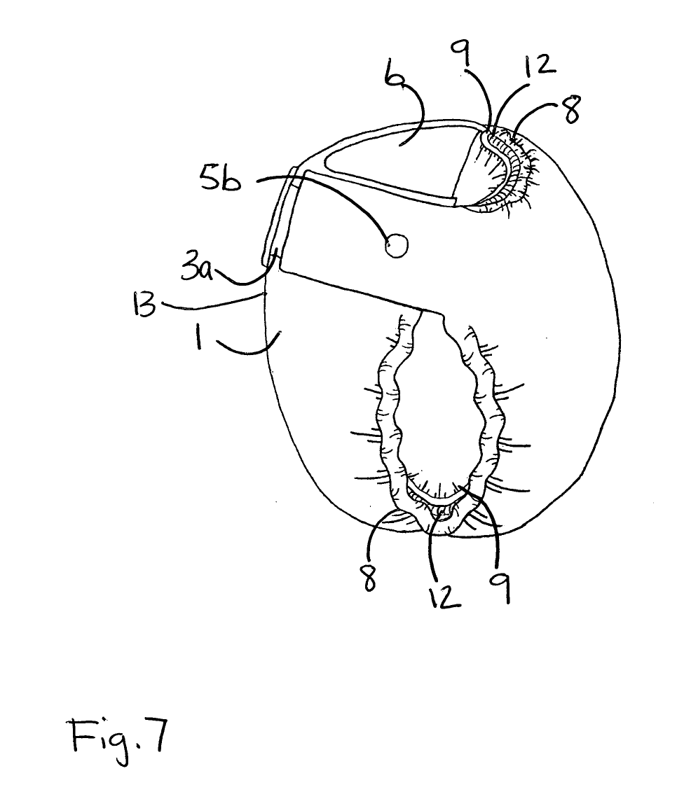

FIG. 8 is a cross sectional view of the fully assembled diaper

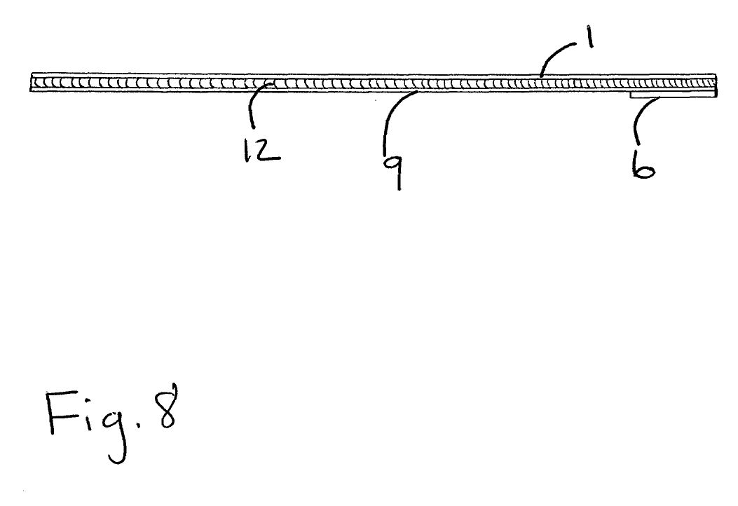

### DRAWINGS—REFERENCE NUMERALS

1—diaper cover outer fabric

2—diaper cover inner fabric

3a—loop tape on tummy panel

3b—loop tape on wings (laundry tabs)

4a—hook tape left wing

4b—hook tape right wing

5a—female snap parts on left wing

5b—female snap parts on right wing

6—flap (inside tummy panel)

7—female snap parts on flap

8—elastic (internal diaper cover)

9—topsheet

10—fold over elastic (FOE) on topsheet

11a—front male snaps on topsheet

11b—back male snaps on topsheet

12—absorbent layer

13—front tummy panel

14a—left wing

14b—right wing

### DETAILED DESCRIPTION—FIRST EMBODIMENT

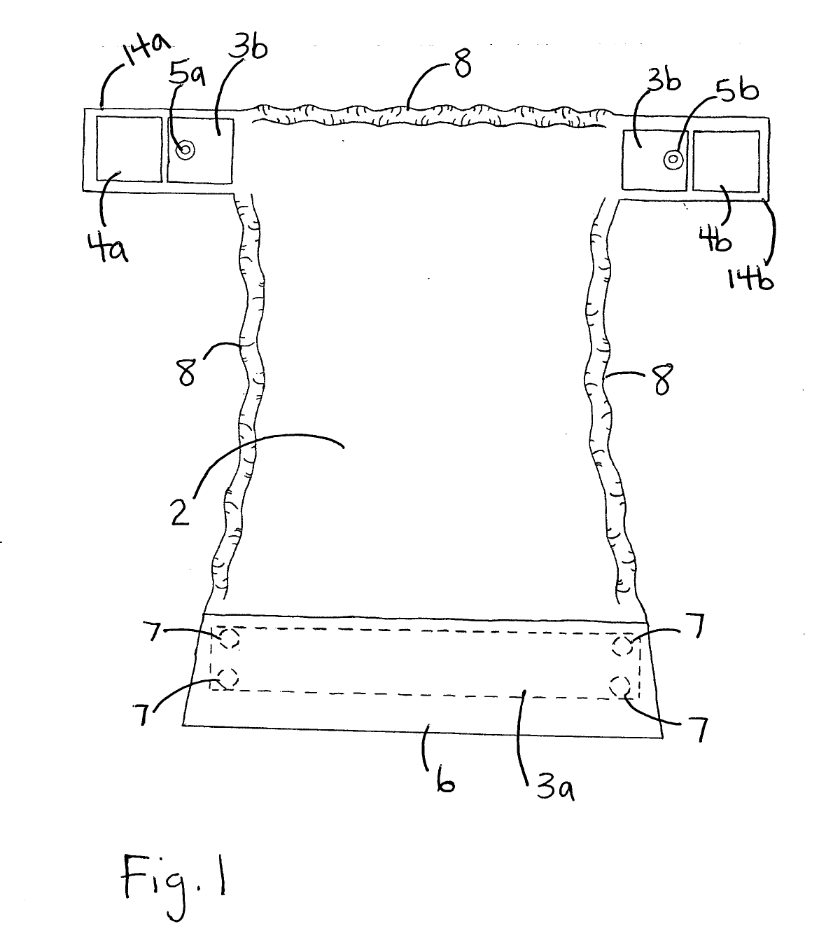

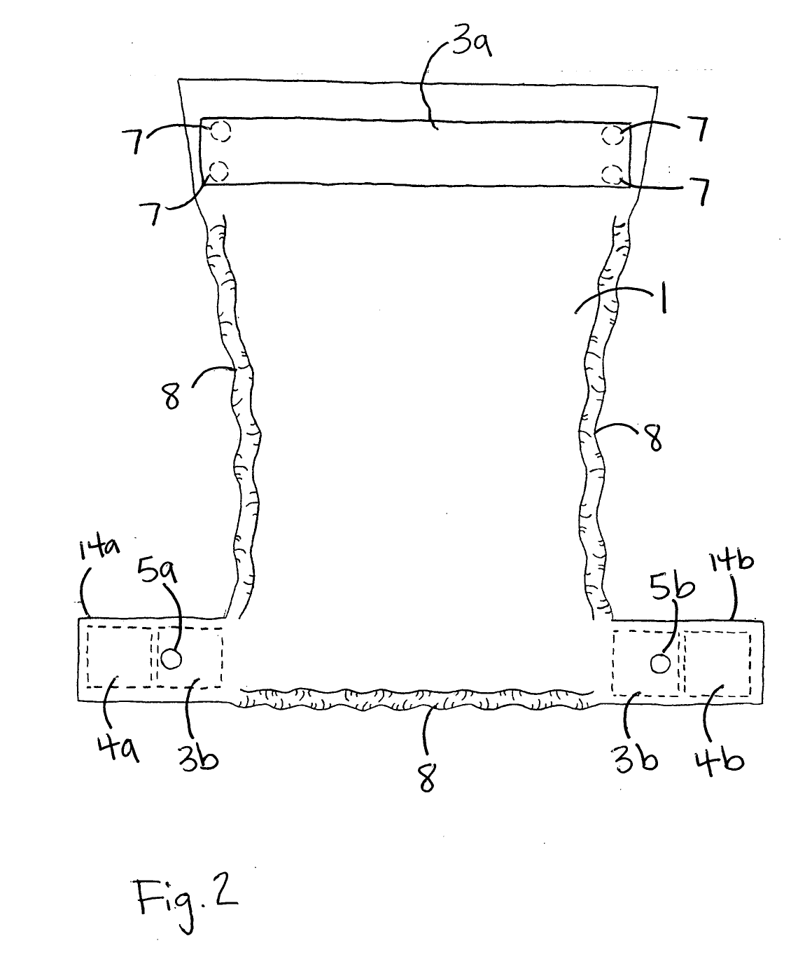

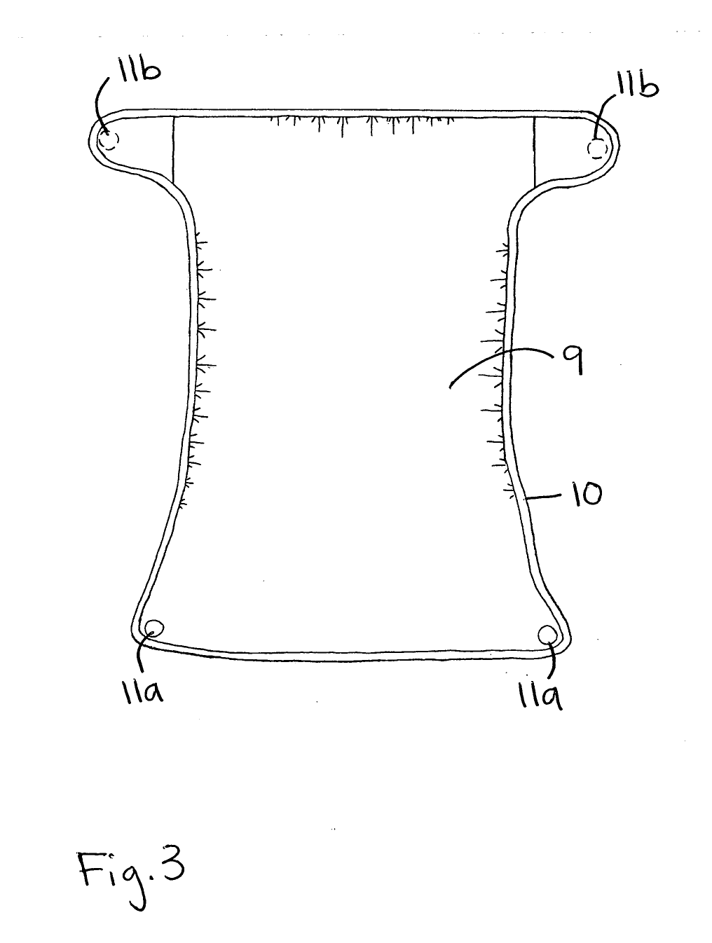

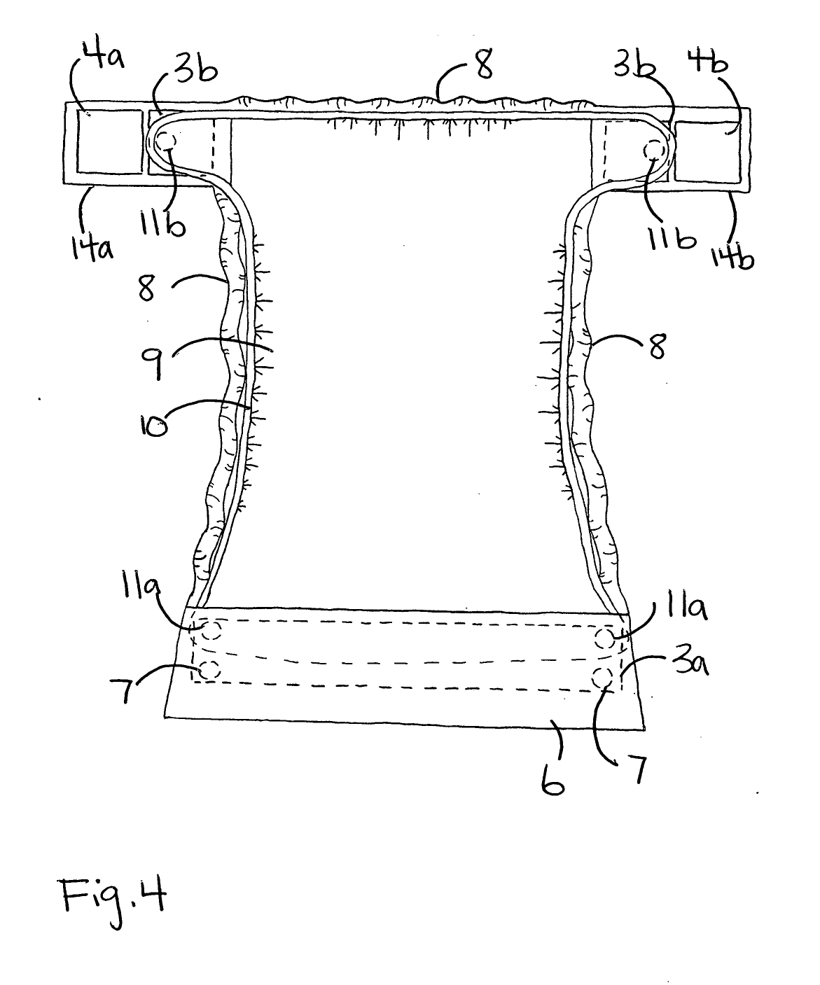

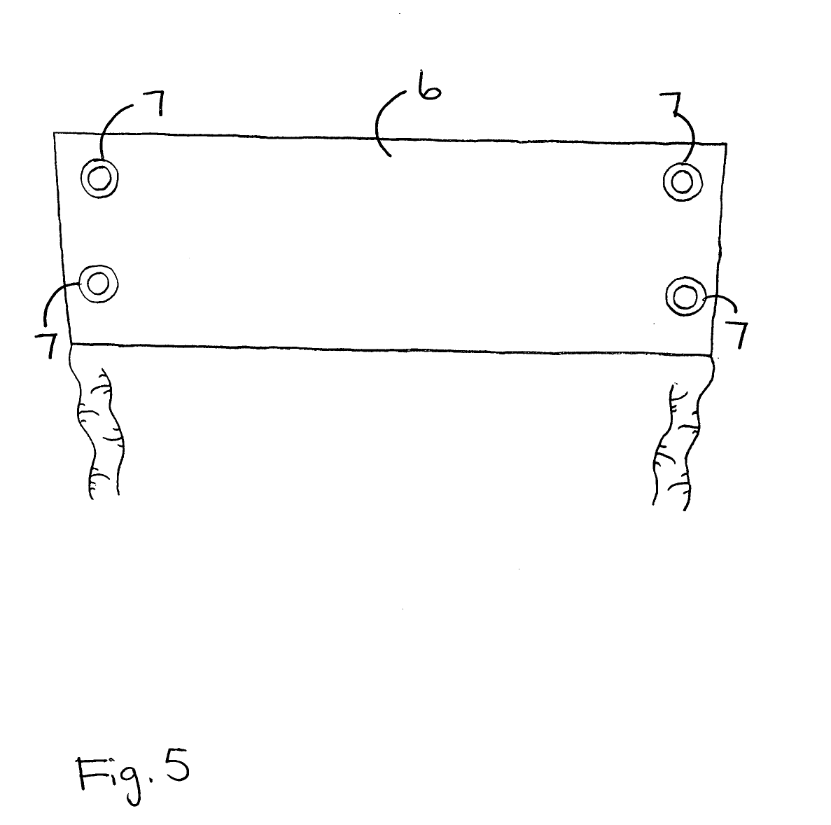

In the currently preferred embodiment, the diaper consists of a moisture-impervious fabric outer layer 1, having a typically hourglass shape to conform to the anatomy of the wearer's body. Elongated tabs at the rear outer edges provide a means for wrapping the diaper around the body and securing hook tapes 4a and 4b to loop tape 3a, securing the diaper to itself. A non-absorbent fabric inner layer 2, having an identical shape is completely attached to the outer layer 1, and elastic 8 is attached internally in appropriate portions of the diaper body to provide an adjustable fit at the leg and waist openings. A flap of moisture-impervious fabric 6 corresponding to approximately one-third of the length of the front panel is attached to the top and side edges inside the diaper cover, concealing female snap parts 7, FIGS. 1 and 2. Two sets of female snap parts 7 are provided for the adjustability of fit for the wearer FIG. 5. A moisture-pervious topsheet 9 is provided, comprising a roughly identical hourglass shape and having a nearly identical length and width as the diaper body FIG. 3. The topsheet is bound in fold-over-elastic 10 and gathered in the curved portions of the hourglass shape to provide adjustable fit in the leg area FIG. 3. The topsheet is attached to the diaper cover by means of male snap parts 11a and 11b located in the four corners of the hourglass shape which attach to female snap parts 5a, 5b, on the elongated tabs of the diaper cover and female snap parts 7 which are concealed in the front flap 6 of the diaper cover FIG. 4.

When the diaper is removed, loop tape 3b is provided on each tab to correspond to hook tapes 4a and 4b, protecting the fabric from being damaged during laundering FIG. 1.

### Operation

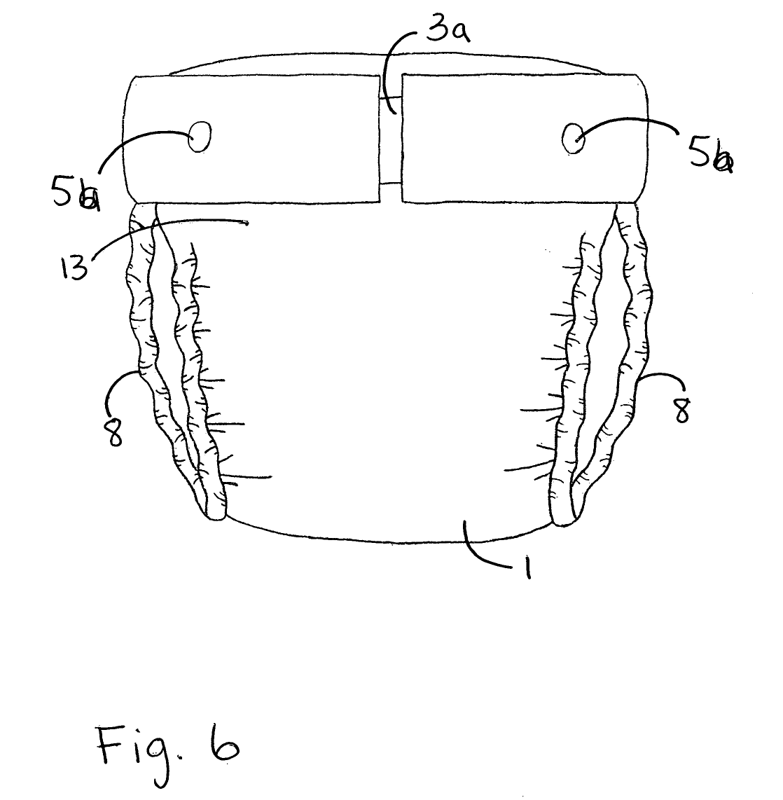

In use, the user will attach the front male snap parts 11a on topsheet 9 to female snap parts 7 on flap 6 of the diaper cover. An absorbent layer 12 with a typical rectangular or hourglass shape (not shown) will be placed between front flap 6 and inner diaper fabric 2 and laid across inner diaper fabric 2. The user then attaches back male snap parts 11b of topsheet 9 to female snap parts 5a and 5b on wings of the diaper cover. The diaper is then placed beneath the wearer with topsheet 9 touching the wearers body. The front tummy panel 13 of the diaper which has front flap 6 and loop tape 3a attached, is then folded between the legs and brought into position over the stomach of the child. Loop tape 3a is provided on the outer fabric of the tummy panel 13, thus when the diaper is folded in the use position FIG. 6, the loop tape 3a appears on the outer top front surface of the diaper. In order to fasten the diaper on the wearer, the wings 14a and 14b are folded into overlapping relationship, so that the hook tape 4a and 4b engages and releasably locks with loop tape 3a.

### Description—Alternative Embodiment

An additional embodiment consists of the diaper cover and topsheet described but replaces loop tape 3a with a plurality of female snap parts and replaces hoop strips 4a and 4b with a plurality of male snap parts, or vice versa.

An additional embodiment consists of the forgoing diaper construction but fastens the diaper at the side by means of female snap parts of each outer edge of the front panel for a total of four female snaps, and a plurality of male snap parts are evenly arranged on the rear wing tabs, or vice versa. The rear wing tabs are wrapped around the wearer and the snap parts secure the diaper to itself.

Alternative embodiments may include a non-elasticized topsheet, a 1-layer diaper cover bound in fold-over-elastic, a diaper cover without a front flap installed where-in the fastening means of attaching the topsheet would be directly on the diaper cover portion, a topsheet consisting of more than one layer of moisture-pervious material, and a topsheet that is secured by hook and loop tape instead of snap fasteners.

### Conclusion, Ramifications, and Scope

Accordingly, the reader will see that the diaper cover with detachably attached topsheet provides and excellent fit, adjustable absorbency, is easy to clean thoroughly, and assures that the wearer's skin is protected both from moisture as well as minimizing direct contact with the topsheet fastening means. Furthermore, the topsheet has the additional advantage of protecting the wearer from being scratched by the edges of the hook and loop tape closures on the wing tabs of the diaper cover.

While the above description contains many specificities, these should not be construed as limitations on the scope of any embodiment, but as merely providing illustration of some of the presently preferred embodiments thereof. Many other ramifications and variations are possible within the teachings of the various embodiments, for example, the detachably attached closures connecting the diaper cover and topsheet may be hook and loop tape, hooks or buttons; the topsheet may be left unelasticized; the number and placement of snap members underneath the front flap may be adjusted, or hook and loop may be used, etc.

Thus the scope of the embodiments should be determined by the appended claims and their legal equivalents, and not by the examples given.

I claim:

1. A reusable diapering system comprising
a: a moisture-impervious outer cover in substantially hourglass shape containing fastener means
b: a detachably attached reusable moisture-pervious topsheet in substantially hourglass shape containing fastener means
c: a means by which said detachably attached topsheet attaches to said moisture-impervious outer cover creating a space between said cover and said topsheet whereby an absorbent material may be placed.

2. The diaper of claim 1 wherein said detachably attached reusable moisture-pervious topsheet is elasticized in the leg and back portions.

3. The diaper of claim 1 wherein said moisture-impervious outer cover contains elastomeric material in the leg and back portions of said cover.

4. The diaper of claim 1 wherein said moisture-pervious topsheet substantially conforms in shape to said moisture-impervious outer cover.
